(19) United States
(12) Patent Application Publication
(10) Pub. No.: US 2019/0030023 A1
(43) Pub. Date: Jan. 31, 2019

(54) METHODS FOR TREATING CANCER

(71) Applicant: X4 Pharmaceuticals, Inc., Cambridge, MA (US)

(72) Inventors: Robert D. Arbeit, West Newton, MA (US); Paula Marie Ragan, Belmont, MA (US)

(21) Appl. No.: 16/070,991

(22) PCT Filed: Jan. 23, 2017
(86) PCT No.: PCT/US17/14578

§ 371 Date: Jul. 18, 2018

Related U.S. Application Data
(60) Provisional application No. 62/281,962, filed on Jan. 22, 2016.

(51) Int. Cl. A61K 31/4709; A61K 39/395; A61P 35/04; A61K 9/48

(52) U.S. Cl. CPC A61K 31/4709; A61K 39/3955; A61K 9/4858; A61K 9/485; A61P 35/04

(57) ABSTRACT

The present invention relates to methods of treating patients with advanced forms of cancer, such as metastatic melanoma and non-small cell lung cancer, in which X4P-001 is administered as monotherapy or in combination with immune checkpoint inhibitors, such as pembrolizumab. The methods demonstrate surprising results, including regression of disease, with comparatively little toxicity.

### CROSS-REFERENCE TO RELATED APPLICATIONS

This application claims the benefit of priority to U.S. Provisional Patent Application Ser. No. 62/281,962, filed Jan. 22, 2016, the entirety of which is hereby incorporated by reference.

### FIELD OF THE INVENTION

The present invention relates to methods for treating cancer, in particular, methods for treatment of patients with advanced melanoma, such as resectable and unresectable melanoma.

### BACKGROUND OF THE INVENTION

Cutaneous malignant melanoma is the fifth most common cancer in men and the sixth most common cancer in women in the United States, with an estimated 73,870 new cases and 9,940 deaths expected in 2015. When discovered early, melanoma is highly curable with 10-year overall survival rates approaching 95% for stage I melanoma and 45-77% for stage II melanoma after complete surgical resection of the primary melanoma. However, surgical treatment may not be feasible for all patients with advanced melanoma. Patients with unresectable or metastatic disease receive systemic treatment, including immunotherapy (e.g. checkpoint inhibitors (CPI) such as anti-PD-1 and anti-CTLA-4 antibodies) and targeted therapy (e.g. BRAF and/or MEK inhibitors for patients with known genetic mutations). Both checkpoint inhibitor immunotherapy and targeted therapy prolong progression-free survival and overall survival.

Moreover, 30% of patients who have undergone complete resection of their primary melanoma will develop local, in-transit and/or nodal recurrence of their disease. In addition, 10% of melanoma patients present with nodal metastases. Among these stage III patients, complete surgical removal is the main treatment for those with resectable disease; however, the risk of recurrence after surgery is very high. Adjuvant therapies with immunomodulating drugs such as high dose interferon-α and the anti-CTLA-4 antibody ipilimumab have shown to improve the recurrence-free survival in patients with resectable stage III melanoma. The impact of these adjuvant treatments on overall survival is not established.

The benefit of neoadjuvant chemo- and immunotherapy has been demonstrated in several operable cancers. Compared to adjuvant therapy, neoadjuvant therapy in patients with locally and regionally advanced cancer has several potential benefits:
Reducing the size of the primary and metastatic tumor increases the probability of achieving negative margin resection.
Tumor exposure to potentially effective systemic therapy is increased while blood and lymphatic vessels remain intact.
Collection of pre- and intra-operative samples of tumor tissue following neoadjuvant therapy offers real-time, in vivo assessment of the effects of the therapy on the tumor cells, the tumor microenvironment (TME), and the immune system.

### DETAILED DESCRIPTION OF CERTAIN EMBODIMENTS OF THE INVENTION

CXCR4 (C-X-C chemokine receptor type 4) is a chemokine receptor expressed on a wide range of cell types, including normal stem cells, hematopoietic stem cells (HSC), mature lymphocytes, and fibroblasts [1]. CXCL12 (previously referred to as SDF-1α) is the sole ligand for CXCR4. The primary physiologic functions of the CXCL12/CXCR4 axis include the migration of stem cells both during embryonic development (CXCR4−/− knock-out embryos die in utero) and subsequently in response to injury and inflammation. Increasing evidence indicates multiple potential roles for CXCR4/CXCL12 in malignancy. Direct expression of one or both factors has been observed in several tumor types. CXCL12 is expressed by cancer-associated fibroblast (CAFs) and is often present at high levels in the TME. In clinical studies of a wide range of tumor types, including breast, ovarian, renal, lung, and melanoma, expression of CXCR4/CXCL12 has been associated with a poor prognosis and with an increased risk of metastasis to lymph nodes, lung, liver and brain, which are sites of CXCL12 expression [2]. CXCR4 is frequently expressed on melanoma cells, particularly the CD133+ population that is considered to represent melanoma stem cells [2, 3] and in vitro experiments and murine models have demonstrated that CXCL12 is chemotactic for those cells [4].

Pembrolizumab is a humanized IgG4 kappa monoclonal antibody that blocks the interaction between PD-1 and its ligands, PD-L1 and PD-L2 [11]. It belongs to the emerging class of immunotherapeutics referred to as checkpoint modulators (CPM). These agents have been developed based on observations that in multiple types of malignancies, the tumor suppresses the host anti-tumor immune response by exploiting counter-regulatory mechanism that normally act as “checkpoints” to prevent the overactivation of the immune system in infection and other situations. In the case of melanoma, PD-L1 is expressed by cells in the TME, engages PD-1, a membrane-associated receptor on CD8+ effector T cells, and triggers inhibitory signaling that reduces the killing capacity of cytotoxic T cells.

Pembrolizumab is currently FDA approved for the treatment of unresectable or metastatic melanoma. In a Phase 3 trial, the objective response rate was 33% compared to 12% for ipilimumab (P<0.001) [11]. Analysis of tumor samples before and during treatment in an earlier study demonstrated that a clinical response was associated with an increase in the density of CD8+ T cells in the tumor parenchyma (center), while disease progression was associated with persistent low levels of those cells [12]. In an autochthonous murine model of pancreatic adenocarcinoma, persistent tumor growth despite administration of anti-PD-L1 was similarly associated failure of tumor-specific cytotoxic T cells to enter the TME despite their presence in the peripheral circulation [7]. This immunosuppressed phenotype was associated with CXCL12 production by CAF. Moreover, administration of a CXCR4 antagonist (AMD3100) induced rapid T-cell accumulation among the cancer cells and, in combination with anti-PD-L1, synergistically decreased tumor growth.

Multiple observations implicate the CXCL12/CXCR4 axis in contributing to the lack (or loss) of tumor responsiveness to angiogenesis inhibitors (also referred to as “angiogenic escape”). In animal cancer models, interference with CXCR4 function has been demonstrated to disrupt the tumor microenvironment (TME) and unmask the tumor to immune attack by multiple mechanisms, including eliminating tumor re-vascularization [19, 20] and increasing the ratio of CD8+ T cells to Treg cells [19, 21,22]. These effects result in significantly decreased tumor burden and increased overall survival in xenograft, syngeneic, as well as transgenic, cancer models [19, 21, 20].

X4P-001, formerly designated AMD11070, is a potent, orally bioavailable CXCR4 antagonist [23], that has demonstrated activity in solid and liquid tumor models [24, and unpublished data] and has previously (under the designations AMD070 and AMD11070) been in Phase 1 and 2a trials involving a total of 71 healthy volunteers [23,25,26] and HIV-infected subjects [27,28]. These studies demonstrated that oral administration of up to 400 mg BID for 3.5 days (healthy volunteers) and 200 mg BID for 8-10 days (healthy volunteers and HIV patients) was well-tolerated with no pattern of adverse events or clinically significant laboratory changes. These studies also demonstrated pharmacodynamic activity, with dose- and concentration-related changes in circulating white blood cells (WBCs); and a high volume of distribution (VL), suggesting high tissue penetrance.

Plerixafor (formerly designated AMD3100, now marketed as Mozobil®) is the only CXCR4 antagonist currently FDA approved. Plerixafor is administered by subcutaneous injection and is approved for use in combination with granulocyte-colony stimulating factor (G-CSF) to mobilize hematopoietic stem cells (HSCs) to the peripheral blood for collection and subsequent autologous transplantation in patients with non-Hodgkin's lymphoma (NEIL) and multiple myeloma (MM).

Both X4P-001 and plerixafor have been studied in murine models of melanoma, renal cell carcinoma, and ovarian cancer and have demonstrated significant anti-tumor activity, including decreased metastasis and increased overall survival [6]. The treatment effect has been associated with decreased presence of myeloid-derived suppressor cells (MDSCs) in the TME and increased presence of tumor-specific CD-8+ effector cells [7, 8].

Without wishing to be bound by any particular theory, it is believed that administration of X4P-001 will increase the density of CD8+ T cells among the melanoma tumor cells and that this effect will be sustained when X4P-001 is given in combination with pembrolizumab. Because X4P-001 is well-tolerated in the body, and may increase the ability of the body to mount a robust anti-tumor immune response, administering X4P-001 in combination with checkpoint modulators in multiple tumor types may substantially increase the objective response rate, the frequency of durable long-term responses, and overall survival.

It is further believed that such a result would be achieved with comparatively little toxicity since CXCR4-targeted drugs would not be expected to induce cell cycle arrest in bone marrow and other normal proliferating cell populations. Accordingly, the present invention provides significant advantages in treatment outcomes utilizing the low toxicity and effects of the CXCR4 inhibitor AMD11070 (X4P-001) on MDSC trafficking, differentiation, and tumor cell gene expression in RCC.

It has now been found that CXCR4 antagonism by X4P-001 provides significant effects which may provide significant treatment benefits in patients with advanced melanoma and other cancers by multiple mechanisms. In certain embodiments, administration of X4P-001 increases the density of CD8+ T cells, thereby resulting in increased anti-tumor immune attack. In certain embodiments, administration of X4P-001 additionally sustains decreases in neoangiogenesis and tumor vascular supply; and interferes with the autocrine effect of increased expression by tumors of both CXCR4 and its only ligand, CXCL12, thereby potentially reducing cancer cell metastasis.

In the present invention, patients with advanced forms of cancer, including melanoma, such as metastatic melanoma, or lung cancer, such as metastatic non-small cell lung cancer, are treated with X4P-001, either as a single agent (monotherapy), or in combination with an immune checkpoint inhibitor, such as pembrolizumab. Pembrolizumab is an antibody to PD-1, which binds to the programmed cell death 1 receptor (PD-1), preventing the receptor from binding to the inhibitory ligand PDL-1, and overrides the ability of tumors to suppress the host anti-tumor immune response, dubbed an immune checkpoint inhibitor.

Without wishing to be bound by any particular theory, it is believed that by combining the two medicaments, the patients' treatment outcome can be further improved by increasing the body's ability to mount a robust anti-tumor immune response.

In some embodiments, X4P-001, or a pharmaceutically acceptable salt thereof, is administered to a patient in a fasted state.

In some embodiments, the present invention provides a method for treating patients with cancer that presents as a solid tumor, particularly melanoma. In some embodiments, the patient has resectable melanoma, meaning that the patient's melanoma is deemed susceptible to being removed by surgery. In other embodiments, the patient has unresectable melanoma, meaning that it has been deemed not susceptible to being removed by surgery.

In some embodiments, the present invention provides a method for treating advanced cancer, such as melanoma or non-small cell lung cancer, in a patient in need thereof comprising administering X4P-001, or a pharmaceutically acceptable salt and/or composition thereof. In certain embodiments, the patient was previously administered an immune checkpoint inhibitor. In some embodiments, the patient was previously administered an immune checkpoint inhibitor selected from the group onsisting of pembrolizumab (Keytruda®, Merck), ipilumumab (Yervoy®, Bristol-Myers Squibb); nivolumab (Opdivo®, Bristol-Myers Squibb) and atezolizumab (Tecentriq®, Genentech).

In certain embodiments, the present invention provides a method for treating cancer in a patient in need thereof, wherein said method comprises administering to said patient X4P-001 in combination with an immunotherapeutic drug, and, in particular, an immune checkpoint inhibitor. In certain embodiments, the X4P-001 and the checkpoint inhibitor are administered simultaneously or sequentially. In certain embodiments, X4P-001 is administered prior to the initial dosing with the immune checkpoint inhibitor. In certain embodiments, the immune checkpoint inhibitor is administered prior to the initial dosing with X4P-001.

In certain embodiments, the immune checkpoint inhibitor is selected from a PD-1 antagonist, a PD-L1 antagonist, and a CTLA-4 antagonist. In some embodiments, X4P-001 is administered in combination with an immunotherapeutic drug selected from the group consisting of ipilimumab (Yervoy®, Bristol-Myers Squibb); atezolizumab (Tecentriq®, Genentech); nivolumab (Opdivo®, Bristol-Myers Squibb) and pembrolizumab (Keytruda®, Merck). In a particular embodiments of the invention, X4P-001 is administered in combination with pembrolizumab (Keytruda®, Merck), previously known as MK-3475.

Other immune checkpoint inhibitors in development may also be suitable for use in combination with X4P-001. These include atezolizumab (Tecentriq®, Genentech/Roche), also known as MPDL3280A, a fully humanized engineered antibody of IgG1 isotype against PD-L1, in clinical trials for non-small cell lung cancer, and advanced bladder cancer, such as advanced urothelial carcinoma; and as adjuvant therapy to prevent cancer from returning after surgery; durvalumab (Astra-Zeneca), also known as MED14736, in clinical trials for metastatic breast cancer, multiple myeloma, esophageal cancer, myelodysplastic syndrome, small cell lung cancer, head and neck cancer, renal cancer, glioblastoma, lymphoma and solid malignancies; pidilizumab (CureTech), also known as CT-011, an antibody that binds to PD-1, in clinical trials for diffuse large B-cell lymphoma and multiple myeloma; avelumab (Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer and gastric cancer; and PDR001 (Novartis), an inhibitory antibody that binds to PD-1, in clinical trials for non-small cell lung cancer, melanoma, triple negative breast cancer and advanced or metastatic solid tumors.

Pembrolizumab (Keytruda®, Merck) is a humanized antibody that targets the programmed cell death (PD-1) receptor. The structure and other properties of pembrolizumab are specified at http://www.drugbank.ca/drugs/DB09037, accessed on Jan. 18, 2016, the disclosure of which is hereby incorporated herein. Pembrolizumab is approved for use in treating unresectable melanoma and metastatic melanoma, and metastatic non-small cell lung cancer in patients whose tumors express PD-1, and have failed treatment with other chemotherapeutic agents. Additionally, pembrolizumab has been tested or mentioned as a possible treatment in other oncologic indications, including solid tumors, thoracic tumors, thymic epithelial tumors, thymic carcinoma, leukemia, ovarian cancer, esophageal cancer, small cell lung cancer, head and neck cancer, salivary gland cancer, colon cancer, rectal cancer, colorectal cancer, urothelial cancer, endometrial cancer, bladder cancer, cervical cancer, hormone-resistant prostate cancer, testicular cancer, triple negative breast cancer, renal cell and kidney cancer, pancreatic adenocarcinoma and pancreatic cancer, gastric adenocarcinoma, gastrointestinal and stomach cancer; brain tumor, malignant glioma, glioblastoma, neuroblastoma, lymphoma, sarcoma, mesothelioma, respiratory papilloma, myelodysplastic syndrome and multiple myeloma.

In a Phase 3 trial in unresectable or metastatic melanoma, the objective response rate was 33% compared to 12% for ipilimumab (P<0.001) [11]. Analysis of tumor samples before and during treatment in an earlier study demonstrated that a clinical response was associated with an increase in the density of CD8+ T cells in the tumor parenchyma (center), while disease progression was associated with persistent low levels of those cells [12]. In an autochthonous murine model of pancreatic adenocarcinoma, persistent tumor growth despite administration of anti-PD-L1 was similarly associated failure of tumor-specific cytotoxic T cells to enter the TME despite their presence in the peripheral circulation [7]. This immunosuppressed phenotype was associated with CXCL12 production by CAF. By increasing the density of CD8+ T cells among the melanoma tumor cells administration of X4P-001 in combination with pembrolizumab or other checkpoint modulators in multiple tumor types may substantially increase the objective response rate, the frequency of durable long-term responses, and overall survival.

In its current prescribed labeling for unresectable or metastatic melanoma, the recommended course of administration for pembrolizumab is 2 mg/kg as an intravenous infusion over 30 minutes every three weeks. In the discretion of the clinician, depending upon individual tolerance, the prescribed dose of pembrolizumab may be increased to 10 mg/kg every 21 days or or 10 mg/kg every 14 days. In the discretion of the clinician, together with the warnings provided with prescribing information, administration of pembrolizumab may be discontinued, or the dose reduced in the case of significant adverse effects.

In some embodiments, the present invention provides a method for treating metastatic melanoma in a patient comprising administering to the patient X4P-001 or a pharmaceutically acceptable salt thereof in combination with an immune checkpoint inhibitor. In some embodiments, the melanoma is resectable and metastatic. In other embodiments, the melanoma is unresectable and metastatic. In some embodiments, the immune checkpoint inhibitor is pembrolizumab.

In some embodiments, the present invention provides a method for treating resectable metastatic melanoma in a patient comprising administering to the patient X4P-001 or a pharmaceutically acceptable salt thereof in combination with an immune checkpoint inhibitor. After completion of treatment in accordance with the present invention, resection surgery may be performed. In other embodiments, the present invention provides a method for treating unresectable metastatic melanoma in a patient comprising administering to the patient X4P-001 or a pharmaceutically acceptable salt thereof in combination with an immune checkpoint inhibitor. In some embodiments, the immune checkpoint inhibitor is pembrolizumab. After completion of treatment in accordance with the present invention, the patient may continue to receive standard of care (SOC) therapy with pembrolizumab or another therapy per the treating clinician's discretion, and such treatment may include further treatment with X4P-001.

In some embodiments, the present invention provides a method for treating a refractory cancer in a patient in need thereof, wherein said method comprises administering to said patient X4P-001 or a pharmaceutically acceptable salt thereof in combination with an immune checkpoint inhibitor. In some embodiments, the refractory cancer is metastatic non-small cell lung cancer (NSCLC) that expresses PD-L1, and which exhibits disease progression after platinum-containing chemotherapy. In some embodiments, the refractory cancer is metastatic NSCLC and the immune checkpoint inhibitor is pembrolizumab.

In some embodiments, a provided method comprises administering the X4P-001, or a pharmaceutically acceptable salt thereof, to a patient in a fasted state and administering the immune checkpoint inhibitor to a patient in either a fasted or fed state.

In certain embodiments, the present invention provides a method for treating cancer in a patient in need thereof, wherein said method comprises administering to said patient X4P-001 or a pharmaceutically acceptable salt thereof in combination with an immune checkpoint inhibitor, further comprising the step of obtaining a biological sample from the patient and measuring the amount of a disease-related biomarker. In some embodiments, the biological sample is a blood sample. In certain embodiments, the disease-related biomarker is circulating CD8+ cells and/or plasma levels of PD-1 and/or PDL-1.

in certain embodiments, the present invention provides a method for treating advanced cancer, such as melanoma or non-small cell lung cancer, in a patient in need thereof, wherein said method comprises administering to said patient X4P-001 or a pharmaceutically acceptable salt thereof in combination with pembrolizumab, further comprising the step of obtaining a biological sample from the patient and measuring the amount of a disease-related biomarker. In some embodiments, the biological sample is a blood sample. In certain embodiments, the disease-related biomarker is circulating CD8+ cells and/or plasma levels of PD-1 and/or PDL-1.

In other embodiments of the invention, X4P-001 or a pharmaceutically acceptable salt thereof is administered in combination with an immune checkpoint inhibitor. The immune checkpoint inhibitor may be an antibody to PD-1, PDL-1, or CTLA-4. In certain embodiments, the immune checkpoint antagonist is selected from the group consisting of pembrolizumab, nivolumab, and ipilimumab.

In some embodiments, the present invention provides a method of treating cancer in a patient in need thereof, wherein said method comprises administering to said patient X4P-001 or a pharmaceutically acceptable salt thereof in combination with an immune checkpoint inhibitor, wherein the X4P-001 or a pharmaceutically acceptable salt thereof and the immune checkpoint inhibitor act synergistically. One of ordinary skill in the art will appreciate that active agents (such as X4P-001 and an immune checkpoint inhibitor) act synergistically when the combination of active agents results in an effect that is greater than additive. In some embodiments, the immune checkpoint inhibitor is pembrolizumab.

### Dosage and Formulations

X4P-001 is a CXCR4 antagonist, with molecular formula C21H27N5; molecular Weight 349.48 amu; appearance white to pale yellow solid; solubility: X4P-001 is freely soluble in the pH range 3.0 to 8.0 (>100 mg/mL), sparingly soluble at pH 9.0 (10.7 mg/mL) and slightly soluble at pH 10.0 (2.0 mg/mL). X4P-001 is only slightly soluble in water; and melting point of 108.9° ΔC.

The chemical structure of X4P-001 is depicted below.

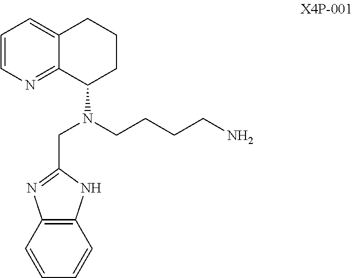 [SMILES: NCCCCN(Cc1nc2ccccc2[nH]1)[C@H]1CCCc2cccnc21]

In certain embodiments, the composition containing X4P-001 is administered orally, in an amount from about 200 mg to about 1200 mg daily. In certain embodiments, the dosage composition may be provided twice a day in divided dosage, approximately 12 hours apart. In other embodiments, the dosage composition may be provided once daily. The terminal half-life of X4P-001 has been generally determined to be between about 12 to about 24 hours, or approximately 14.5 hrs. Dosage for oral administration may be from about 100 mg to about 1200 mg once or twice per day. In certain embodiments, the dosage of X4P-001 useful in the invention is from about 200 mg to about 600 mg daily. In other embodiments, the dosage of X4P-001 useful in the invention may range from about 400 mg to about 800 mg, from about 600 mg to about 1000 mg or from about 800 mg to about 1200 mg daily. In certain embodiments, the invention comprises administration of an amount of X4P-001 of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg or about 1600 mg.

In some embodiments, a provided method comprises administering to the patient a pharmaceutically acceptable composition comprising X4P-001 wherein the composition is formulated for oral administration. In certain embodiments, the composition is formulated for oral administration in the form of a tablet or a capsule. In some embodiments, the composition comprising X4P-001 is formulated for oral administration in the form of a capsule.

In certain embodiments, a provided method comprises administering to the patient one or more capsules comprising 100-1200 mg X4P-001 active ingredient; and one or more pharmaceutically acceptable excipients.

In certain embodiments, the present invention provides a composition comprising X4P-001, or a pharmaceutically acceptable salt thereof, one or more diluents, a disintegrant, a lubricant, a flow aid, and a wetting agent. In some embodiments, the present invention provides a composition comprising 10-1200 mg X4P-001, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In some embodiments, the present invention provides a unit dosage form wherein said unit dosage form comprises a composition comprising 10-200 mg X4P-001, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In certain embodiments, the present invention provides a unit dosage form comprising a composition comprising X4P-001, or a pharmaceutically acceptable salt thereof, present in an amount of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg or about 1600 mg. In some embodiments, a provided composition (or unit dosage form) is administered to the patient once per day, twice per day, three times per day, or four times per day. In some embodiments, a provided composition (or unit dosage form) is administered to the patient once per day or twice per day.

In some embodiments, the present invention provides a unit dosage form comprising a composition comprising:
(a) X4P-001, or a pharmaceutically acceptable salt thereof—about 30-40% by weight of the composition;
(b) microcrystalline cellulose—about 20-25% by weight of the composition;
(c) dibasic calcium phosphate dihydrate—about 30-35% by weight of the composition;
(d) croscarmellose sodium—about 5-10% by weight of the composition;
(e) sodium stearyl fumarate—about 0.5-2% by weight of the composition;
(f) colloidal silicon dioxide—about 0.1-1.0% by weight of the composition; and
(g) sodium lauryl sulfate—about 0.1-1.0% by weight of the composition.

In some embodiments, the present invention provides a unit dosage form comprising a composition comprising:
(a) X4P-001, or a pharmaceutically acceptable salt thereof—about 37% by weight of the composition;
(b) microcrystalline cellulose—about 23% by weight of the composition;
(c) dibasic calcium phosphate dihydrate—about 32% by weight of the composition;
(d) croscarmellose sodium—about 6% by weight of the composition;
(e) sodium stearyl fumarate—about 1% by weight of the composition;
(f) colloidal silicon dioxide—about 0.3% by weight of the composition; and
(g) sodium lauryl sulfate—about 0.5% by weight of the composition.

Pembrolizumab has been approved by the FDA for treatment of unresectable or metastatic melanoma or metastatic non-small cell lung cancer, and is generally administered at a dosage of 2 mg/kg as an intravenous infusion over 30 minutes once every 3 weeks. Generally, the amount of pembrolizumab or other immune checkpoint inhibitor useful in the present invention will be dependent upon the size, weight, age and condition of the patient being treated, the severity of the disorder or condition, and the discretion of the prescribing physician.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus, in some embodiments, the invention provides a kit that includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### EXEMPLIFICATION

### Example 1

### Measurement of CD8+ T Cells

Assessment of the effectiveness of the present invention can be made in part by measurement of the CD8+ T cell population. Expanding or increasing the density of CD8+ T cells, such as T-infiltrating lymphocytes (TIL), can help increase tumor recognition and ultimately tumor regression. Dudley et al., (2010) Clin. Cancer Research, 16:6122-6131. CD8+ T cells can be detected, isolated and quantified utilizing methods described in Herr et al., (1996), J. Immunol. Methods 191:131-142; Herr et al., (1997) J. Immunol. Methods 203:141-152; and Scheibenbogen et al., (2000) J Immunol. Methods 244:81-89. The full disclosure of each of these publications is hereby incorporated by reference herein.

### Example 2

### Criteria for Evaluating Response in Patients with Solid Tumors

The response of patients with solid tumors to treatment can be evaluated using the criteria set forth in RECIST 1.1, Eisenhauer et al., (2009) Eur. J. Cancer, 45:228-247, the full disclosure of which is hereby incorporated by reference herein.

### Example 3

### Human Melanoma Xenograft Model

In order to assess the effects of the present invention on the presence of human CD8+ effector T cells, accumulation of T_{regs} in the tumor microenvironment and, ultimately, the effects on metastatic melanoma, a human melanoma xenograft model can be used, as described in Spranger et al. (2013) Sci. Transl. Med., 5:200ra116.

### Example 4

### Clinical Treatment Regimen—Resectable or Unresectable Metastatic Melanoma

Treatment with X4P-001 as a monotherapy, or in combination with a checkpoint inhibitor, such as pembrolizumab, may be performed in cycles, such as on a 3 week or 9 week cycle. In certain embodiments, the cycle is 9 weeks long. X4P-001 at a determined dose from 200 mg to 1200 mg daily is administered orally either once daily or twice daily in divided doses. Patients are instructed about both dosing schedule and requirements relating to food or drink near the time of dosing.

Dosing Schedule. The daily dose is taken first thing in the morning. Where the dose is divided, the first daily dose is taken in the morning and the second daily dose approximately 12 hours later using the following guidelines:
Dosing should be at the same time(s) each day±2 hr.
For twice daily dosing, the interval between successive doses should not be <9 hours nor >15 hours. If the interval would be >15 hrs, the dose should be omitted and the usual schedule resumed at the next dose.
Restrictions relating to food. Absorption is impacted by food and patients will be instructed as follows:
For the morning dose
   No food or drink (except water) after midnight until the time of dosing
   No food or drink (except water) for 2 hour after dosing.
For the second daily dose, if applicable
   No food or drink (except water) for 1 hour before dosing
   No food or drink (except water) for 2 hours after dosing.

Pembrolizumab is administered consistent with prescribed labeling information. Concomitant treatment with X4P-001 and pembrolizumab may be administered, beginning with daily administration of X4P-001 at day 1. Initial treatment with pembrolizumab is at 2 mg/kg administered by intravenous infusion over 30 minutes in clinic at the week 4 and 7 visits. Patients may, with the approval of their clinician, vary the dosing schedule or dosage of pembrolizumab.

Dosing of X4P-001 and/or pembrolizumab may be adjusted by the clinician as appropriate. The dose of X4P-001 and/or pembrolizumab may be lowered according to the judgment of the clinician. If a patient receiving X4P-001 in combination with pembrolizumab experiences an adverse event at Grade >2, the dose of X4P-001 and/or pembrolizumab may be lowered according to the judgment of the clinician. If a patient successfully completes the first 4 weeks of treatment, that is, without experiencing any adverse events greater than Grade 2, the daily dose of X4P-001 and/or pembrolizumab may be increased, consistent with the judgment of the clinician.

Patients with resectable metastatic melanoma, after combination treatment with X4P-001 and pembrolizumab, will typically undergo complete resection, or resection that is as complete as possible, and could continue to be monitored for recurrence, and/or undergo standard of care (SOC) treatment. This could mean continued use of pembrolizumab, or it could mean some other treatment at the clinician's discretion. Patients with unresectable metastatic melanoma, after treatment, will continue to undergo SOC treatment. Such SOC treatment may or may not include a further regimen of X4P-001, with or without pembrolizumab.

### Evaluation of Response to Treatment and Disease Status

Baseline radiologic assessment of the patient is conducted in order to confirm whether the patient has resectable disease. At end of treatment, repeat imaging will be performed using the same modality.

At initial assessment, the patient is diagnosed as having malignant melanoma, including Stage III (any substage) or Stage IV (with isolated skin metastasis only). Patient is assessed for cutaneous/subcutaneous lesions, including those that will be biopsied clinically.

Cutaneous/subcutaneous lesions ≥3 mm are assessed clinically by the investigator, including the number, distribution, and a description of the lesions (e.g. nodular, popular, macular, pigmented, etc.). The size of the cutaneous lesions is determined using photographs of the lesions (including a ruler with patient study identification and date) obtained as indicated in the schedule of events. Lymph nodes are examined at each visit and the location and size of palpable nodes recorded.

Clinical assessments of cutaneous/subcutaneous disease are conducted at each of day 1, week 4 and week 7, and as indicated based on new signs, symptoms or laboratory findings. Assessments will include physical examination (including lymph nodes) and photographs of all cutaneous lesions, including a ruler marked with patient study number and date.

Tumor biopsy samples are assessed by routine histology and analyzed for tumor cell markers (e.g., CD-133) and for immune-related biomarkers (see below Table) to determine the effects of CXCR4 antagonism on the inflammatory cell infiltrates and on the tumor cells.

### Exemplary Immune-Related Biomarkers

| Cell Types | Cell Surface Markers |
|---|---|
| Treg | CD4+/CD25hi+/intracellular FOXP3+; or |
| Lymphocytes | CD4+/CD25hi+/CD39+ |
| T cells | CD3+CD4+ |
| | CD3+CD8+ |
| | CD3+CD4+CD25+ |
| | CD137+ |
| Myeloid-derived | Lin12/HLA-DR2/CD33+/CD11b+ lymphoid |
| suppressor cells | (small FSCxSSC) gate or |
| | Lin12/HLA-DR2/CD33+/CD11b+ monocyte |
| | (larger FSCxSSC) gate or |
| | HLA-DR+ lo/CD14+ monocyte gate |

Patients with melanoma are expected to exhibit between 1300±1700 (mean±SD) CD8+ T cells/mm−2 in melanoma tumor parenchymal.

### Pharmacokinetic Assessments

If desired, pharmacokinetic assessment of blood samples for plasma levels of X4P-001 and pembrolizumab may be conducted. Blood samples are collected as scheduled. For example, samples may be taken at day 1, week 4 and week 7. Samples are analyzed for X4P-001 concentration using reversed-phase high performance liquid chromatography (RP-HPLC) with MS/MS detection. The validated range of this bioanalytic method is 30 to 3,000 ng/mL in plasma.

The initial measurement at day 1 is designated as baseline. At week 4 and week 7, measurements of CD8+ T cells are taken and compared to baseline.

A primary comparison is the density of specific cell phenotypes in the tumor microenvironment in the pre-treatment biopsy vs. the Week 4 and EOT biopsies. CD8+ T cells/mm⁻² are measured in melanoma tumor parenchyma prior to treatment. Patients with melanoma are expected to exhibit between 1300±1700 (mean±SD) CD8+ T cells/mm⁻² in melanoma tumor parenchyma prior to treatment. A 100% increase (mean 2600 cells/mm⁻²) at week 4 compared to baseline is considered to be a positive response.

Secondary analyses include (a) comparison of cell phenotypes in the Week 4 vs. EOT biopsies, (b) changes over time in phenotypes among peripheral blood mononuclear cells (PBMCs) and in serum biomarker levels. Normally distributed continuous variables are analyzed using t-test and ANOVA/ANCOVA, as appropriate. Variables whose results are not normally distributed are analyzed by non-parametric statistics. Fisher's exact test is used for categorical variables.

Pharmacokinetic assessment of pembrolizumab may be accomplished using techniques, such as those described in Patnaik et al. (2015) Clin. Cancer Res. 21:4286-4293, the full disclosure of which is hereby specifically incorporated herein by reference.

### REFERENCES

1. Ratajczak, et al. The pleotropic effects of the SDF-1-CXCR4 axis in organogenesis, regeneration, and tumorigenesis. Leukemia 2006:20; 1915-1924.
2. Scala, et al. Expression of CXCR4 predicts poor prognosis in patients with malignant melanoma. Clin Cancer Res 2005:11; 1835-1841.
3. Toyozawa, et al. Chemokine receptor CXCR4 is a novel marker for the progression of cutaneous malignant melanoma. Acta Histochem Cytochem. 2012; 45:293-299.
4. Kim, et al. CXCR4 signaling regulates metastasis of chemoresistant melanoma cells by a lymphatic metastatic niche. Cancer Res. 2010; 70:10411-10421.
5. Mosi R M, Anastassova V, Cox J, et al. The molecular pharmacology of AMD11070: An orally bioavailable CXCR4 HIV entry inhibitor. Biochem Pharmacol. 2012; 83:472-479.
6. D'Alterio, et al. Inhibition of stromal CXCR4 impairs development of lung metastases. Cancer Immunol Immunother. 2012:61; 1713-1720.
7. Feig, et al. Targeting CXCL12 from FAP-expressing carcinoma-associated fibroblasts synergizes with anti-PD-L1 immunotherapy in pancreatic cancer. PNAS 2013; 110:20212-20217.
8. Zhang et al. Preferential involvement of CXCR4 and CXCL12 in T cell migration toward melanoma cells. Cancer Biol Ther. 2006; 5:1034-1312.
9. Stone, et al. Multiple-Dose Escalation Study of the Safety, Pharmacokinetics, and Biologic Activity of Oral AMD070, a Selective CXCR4 Receptor Inhibitor, in Human Subjects. Antimicrob Agents Chemother. 2007; 51(7):2351-2358.
10. Moyle, et al. Proof of Activity with AMD11070, an Orally Bioavailable Inhibitor of CXCR4-Tropic HIV Type 1. Clin Infect Dis. 2009; 48:798-805.
11 Robert, et al. Pembrolizumab versus Ipilimumab in Advanced Melanoma. N Engl J Med. 2015; 372:2521-2532.
12. Tumeh, et al. PD-1 blockade induces responses by inhibiting adaptive immune resistance. Nature 2014:515; 568-571.
13. Tarhini, et al. Immune Monitoring of the Circulation and the Tumor Microenvironment in Patients with Regionally Advanced Melanoma Receiving Neoadjuvant Ipilimumab. PLoS One 2014; 9(2):e87705.
14. Nyunt, et al. Pharmacokinetic Effect of AMD070, an Oral CXCR4 Antagonist, on CYP3A4 and CYP2D6 Substrates Midazolam and Dextromethorphan in Healthy Volunteers. J Acquir Immune Defic Syndr. 2008; 47:559-565.
15 Cao, et al. Effect of Low-Dose Ritonavir on the Pharmacokinetics of the CXCR4 Antagonist AMD070 in Healthy Volunteers. Antimicrob Agents Chemother. 2008; 52:1630-1634.
16. Common Terminology Criteria for Adverse Events (CTCAE). Version 4.0, 28 May 2009. U.S. Department of Health and Human Services, National Institutes of Health, National Cancer Institute. NIH Publication No. 03-5410.
17. NCI CTCAE v4.03, 14 Jun. 2010 available at (accessed 6 Apr. 2015): http://evs.nci.nih.gov/ftp1/CTCAE/CTCAE_4.03_2010-06-14_QuickReference_5x7.pdf
18. WMA Declaration of Helsinki—Ethical Principles for Medical Research Involving Human Subjects. Available at (accessed 6 Apr. 2015) http://www.wma.net/en/30publications/10policies/b3/
19. Vanharanta et al. Epigenetic expansion of VHL-HIF signal output drives multiorgan metastasis in renal cancer. Nat Med 2013; 19: 50-6.
20. Gale and McColl, Chemokines: extracellular messengers for all occasions? BioEssays 1999; 21: 17-28.
21. Highfill et al., Disruption of CXCR2-mediated MDSC tumor trafficking enhances anti-PD1 efficacy. Sci Transl Med 2014; 6: ra67.
22. Facciabene et al., Tumour hypoxia promotes tolerance and angiogenesis via CCL28 and Treg cells. Nature 2011; 475: 226-230.
23. Montane et al., Prevention of murine autoimmune diabetes by CCL22-mediated Treg recruitment to pancreatic islets. J Clin Invest 2011; 121: 3024-8.
24. Acharyya et al., CXCL1 paracrine network links cancer chemoresistance and metastasis. Cell 2012; 150: 165-78.
25. Zhao et al., TNF signaling drives myeloid-derived suppressor cell accumulation. J Clin Invest 2012; 122: 4094-4104.
26. Silva et al., Profiling essential genes in human mammary cells by multiplex RNA1 screening. Science 2008; 319: 617-20.
27. Schlabach et al., Cancer proliferation gene discovery through functional genomics. Science 2008; 319: 620-24.
28. Shen et al., CXCR4-mediated STAT3 activation is essential for CXCL12-induced invasion in bladder cancer. Tumour Biol 2013; 34: 1839-45.

We claim:

1. A method for treating cancer in a patient in need thereof, wherein said method comprises administering to said patient X4P-001 or a pharmaceutically acceptable salt thereof in combination with an immune checkpoint inhibitor.

2. The method of claim 1, wherein the cancer is selected from the group consisting of metastatic melanoma or metastatic non-small cell lung cancer.

3. The method of claim 2, wherein the patient has previously been treated with an immune checkpoint inhibitor.

4. The method of any of claims 1-3, wherein the immune checkpoint inhibitor is pembrolizumab.

5. The method of any of claims 1-3, wherein the patient is treated with X4P-001 or a pharmaceutically acceptable salt thereof in an amount effective to increase CD8+ T cell density, and then receives additional treatment with an immune checkpoint inhibitor.

6. The method of any of claims 1-3, further comprising the step of obtaining a biological sample from the patient and measuring the amount of a disease-related biomarker.

7. The method of claim 4, wherein the biological sample is a blood sample.

8. The method of claim 5, wherein the disease-related biomarker is circulating CD8+ T cells.

9. The method of any of claims 1-6, wherein the X4P-001 or pharmaceutically acceptable salt thereof is administered orally twice per day.

10. A method for increasing responsiveness to treatment with an immune checkpoint inhibitor in a patient receiving said treatment, said method comprising administering to said patient X4P-001 or a pharmaceutically acceptable salt thereof in an amount effective to increase CD8+ T cell density. A unit dosage form comprising a composition comprising:
(a) X4P-001, or a pharmaceutically acceptable salt thereof—about 30-40% by weight of the composition;
(b) microcrystalline cellulose—about 20-25% by weight of the composition;
(c) dibasic calcium phosphate dihydrate—about 30-35% by weight of the composition;
(d) croscarmellose sodium—about 5-10% by weight of the composition;
(e) sodium stearyl fumarate—about 0.5-2% by weight of the composition;
(f) colloidal silicon dioxide—about 0.1-1.0% by weight of the composition; and
(g) sodium lauryl sulfate—about 0.1-1.0% by weight of the composition.

12. The unit dosage form of claim 11, in the form of a capsule.

13. The unit dosage form of claim 12, wherein the capsule comprises about 100 mg X4P-001, or a pharmaceutically acceptable salt thereof.

14. A method for treating metastatic melanoma in a patient in need thereof, comprising the step of administering to the patient the unit dosage form of claim 11 in combination with an immune checkpoint inhibitor.

15. The method of claim 14, wherein the immune checkpoint inhibitor is pembrolizumab.

16. The method of claim 15, wherein the metastatic melanoma is resectable.

17. The method of claim 16, wherein the patient has undergone surgery for removal of some or all of the metastatic melanoma.

18. The method of claim 14, wherein the metastatic melanoma is unresectable.
